(19) United States
(12) Patent Application Publication
(10) Pub. No.: US 2008/0161647 A1
(43) Pub. Date: Jul. 3, 2008

(54) Device and method for multiple illumination fields of an in-vivo imaging device

(76) Inventor: Amit Pascal, Haifa (IL)

Correspondence Address: Pearl Cohen Zedek Latzer, LLP, 1500 Broadway, 12th Floor, New York, NY, 10036, US

(21) Appl. No.: 11/645,797

(22) Filed: Dec. 27, 2006

(51) Int. Cl. A61B 1/06

(52) U.S. Cl. 600/178

(57) ABSTRACT

A device and method for capturing images of the gastrointestinal tract, or other body lumens or cavities of a patient, captured using one or more illumination sources having different fields of illumination.

### FIELD OF THE INVENTION

The present invention relates to multiple illumination fields of an in-vivo imaging device. More specifically, the present invention relates to an in-vivo imaging device having two or more illumination sources which may provide multiple illumination fields.

### BACKGROUND OF THE INVENTION

In-vivo sensing devices for imaging of the gastrointestinal (GI) tract or other body lumens of a patient such as, for example, ingestible imaging capsules, may wirelessly transmit image data to an external data recorder. The data recorder may be affixed to the patient by a strap or a belt so that the patient may freely perform normal actions during an observation period that may begin after swallowing of the in-vivo imaging device and end upon its excretion. The data recorder may have radio communication capability and it may have connected to it one or more antennas for receiving the image data transmitted by the in-vivo imaging device and the data recorder may have a memory for storing the received image data. After the observation period, the patient may deliver the data recorder to an operator, for example, a health professional who may download the stored image data for processing and for performing analysis of the GI tract for diagnosis purposes.

The image data includes images of the GI tract captured by an imager in the in-vivo imaging device as it passes through the GI tract. The image data may be downloaded from the data recorder to a workstation, or the like, where it may undergo various forms of image processing prior to analysis of the images of the GI tract for diagnosis purposes. The images may be obtained using certain field of view and incorporating a matching illumination system, the illumination is achieved by a light source(s) having a certain field of illumination.

### SUMMARY OF THE INVENTION

In accordance with some embodiments of the present invention, there is provided an in vivo imaging device comprising:

an imager; and
a plurality of illumination sources wherein at least one illumination source has a first field of illumination and at least second illumination source has a second field of illumination.

In accordance with some embodiments, said first field of illumination has a spatial illumination angle of substantially 1-90 degrees and wherein said second field of illumination has a spatial illumination angle of substantially 91-180 degrees.

In accordance with some embodiments, the vivo imaging device comprises a controller configured to control the illumination sources in a selective manner.

In accordance with some embodiments, the selective manner comprises activating a first illumination source to illuminate a first target area and activating a second illumination source to illuminate a second target area.

In accordance with some embodiments, said first target area has different characteristics than said second target area.

In accordance with some embodiments, the vivo imaging device further comprises a transmitter for transmitting image data.

In accordance with some embodiments, the illumination sources are located proximal to one end of the device.

In accordance with some embodiments, the illumination sources are located proximal to both ends of the device.

In accordance with some embodiments of the present invention, there is also provided a method providing an in vivo capturing of images, the method comprising: illuminating an in vivo target area with at least one illumination source having a first field of illumination or with at least a second illumination source having a second field of illumination.

In accordance with some embodiments, the method further comprises controlling activating said at least first illumination source or said at least second illumination source.

### BRIEF DESCRIPTION OF THE DRAWINGS

The present invention will be understood and appreciated more fully from the following detailed description taken in conjunction with the drawings in which:

FIG. 1 is a simplified illustration of an in-vivo imaging device according to embodiments of the present invention;

FIG. 2 is an illustrative end view of an in-vivo imaging device in accordance with embodiments of the present invention;

FIG. 3 is a simplified illustrative side view of an in-vivo imaging device with illumination sources at both ends;

FIG. 4 is an illustration of an in-vivo imaging device in the GI tract with in accordance with embodiments of the present invention; and

FIG. 5 is a flowchart depicting a method according to an embodiment of the invention.

It will be appreciated that for simplicity and clarity of illustration, elements shown in the figures have not necessarily been drawn accurately or to scale. For example, the dimensions of some of the elements may be exaggerated relative to other elements for clarity, or several physical components may be included in one functional block or element. Further, where considered appropriate, reference numerals may be repeated among the figures to indicate corresponding or analogous elements.

### DETAILED DESCRIPTION OF THE INVENTION

In the following description, various aspects of the present invention will be described. For purposes of explanation, specific configurations and details are set forth in order to provide a thorough understanding of the present invention. However, it will also be apparent to one skilled in the art that the present invention may be practiced without the specific details presented herein. Furthermore, well-known features may be omitted or simplified in order not to obscure the present invention.

The device and method of the present invention may be used with an imaging system or device such as that described in U.S. Pat. No. 5,604,531 entitled “In Vivo Video Camera System,” which is incorporated herein by reference. A further example of an imaging system and device with which the device and method of the present invention may be used is described in U.S. Pat. No. 7,009,634 entitled “Device for In Vivo Imaging,” which is incorporated herein by reference. For example, a swallowable imaging capsule such as that described in U.S. Pat. No. 7,009,634, may be used in the present invention. A further example of swallowable imaging capsules that may be used with the device and method of the present invention are those described in U.S. patent application Publication No. 2002/0109774 entitled “System and Method Wide Field Imaging of Body Lumens,” which is incorporated herein by reference.

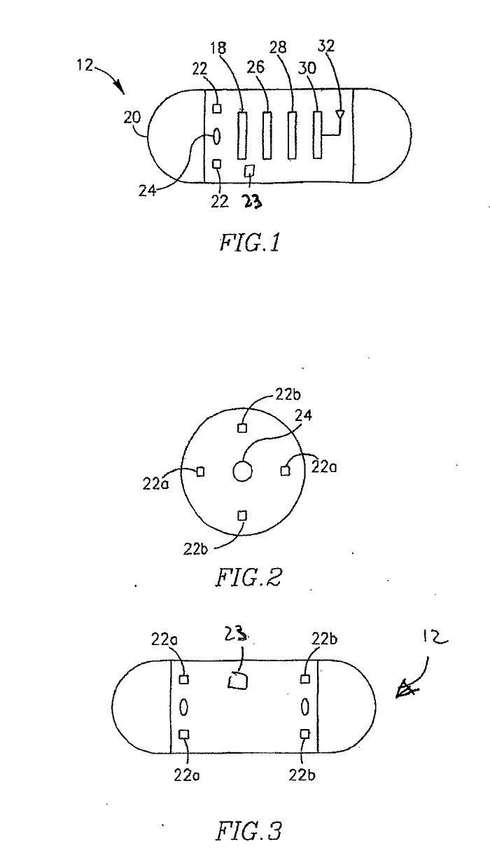

Reference is made to FIG. 1, showing in-vivo imaging device 12 according to embodiments of the present invention. In some embodiments, the in-vivo imaging device 12 may be a wireless device. In some embodiments, the in-vivo imaging device 12 may be autonomous. In some embodiments, the in-vivo imaging device 12 may be a swallowable capsule for imaging the gastrointestinal (GI) tract of a patient. However, other body lumens or cavities may be imaged or examined with the in-vivo imaging device 12.

The in-vivo imaging device 12 may be cylindrical in shape with dome-like ends and may include at least one imager 18 for capturing image data in the form of image frames of images of the gastrointestinal tract or other body lumens or cavities, a viewing window 20 at least one of the ends, one or more illumination sources 22, an optical system 24, a power supply such as a battery 26, a processor 28, a control unit 23, a transmitter 30, and an antenna 32 connected to the transmitter 30. As the in-vivo imaging device 12 traverses the gastrointestinal tract or other body lumens of a patient, it takes a series of images thereof. The illumination sources 22 may be Light Emitting Diodes (LED) or other suitable illumination sources for illuminating a target area from which image frames are to be captured. The target area may be an area of the gastrointestinal tract or other body lumens or cavities of the patient. When viewing certain lumens or cavities, it may be advantageous for the illumination sources to have various fields of illumination. Illumination sources 22 may have multiple illumination fields, for example, wide field illumination and/or narrow field illumination. “Illumination field” as used herein may refer to any angle, direction, orientation or perspective of illumination or a combination thereof, relative to a target area or a viewing site.

Imager 18 of the in-vivo imaging device 12 may capture series of images to form a data stream, forming the frames of a video movie. The imager 18 may be and/or may contain a CMOS imager. Alternatively, other imagers may be used, e.g. a CCD imager or other imagers. The image data and or other data captured by the in-vivo imaging device 12 may be transmitted as a data signal by wireless connection, e.g. by wireless communication channel, by the transmitter 30 via the antenna 32, from the in-vivo imaging device 12 and received by an external recorder.

Control unit 23 may be connected to each of the illumination sources 22 and to imager 18, to synchronize the illumination of the in-vivo site by each of illumination sources 22 with the capturing of images by imager 18 and to control the illumination sources 22 in a selective manner. The control unit 23 may be any sort of device or controller enabling the control of components. For example, a microchip, a microcontroller, or device acting on remote commands may be used.

While in an exemplary embodiment, the illumination produced by the illumination sources 22 may be substantially white light, in alternate embodiments, different illumination may be produced. For example, infra-red, red, blue or green light may be produced. Furthermore, while in one embodiment all illumination sources 22 produce the same spectrum of illumination, in alternate embodiments each of the illumination source may produce different spectra. Each of illumination sources 22 may be, for example, an individual source, such as a lamp or a LED, or may be sets of illumination sources, arranged in a certain configuration such as a ring of LEDs that may be arranged, for example, around optical system 24. Any other illumination source(s) having similar of other arrangements may be used.

Reference is now made to FIG. 2, showing an illustrative end view of an in-vivo imaging device in accordance with embodiments of the present invention. The in-vivo imaging device 12 may have one or more illumination sources 22 (22a, 22b) which may include LEDs, incandescent sources, or other suitable light sources that may enable in-vivo illumination and may be located proximal to or at least one end of the in-vivo imaging device 12. The target area may be an area of the gastrointestinal tract, for example, the stomach, the esophagus or other body lumens or cavities of the patient. The in-vivo imaging device 12 may have one or more types of illumination sources in order to generate multiple available fields of illumination for use with, for example, various target areas. For example, in some embodiments, some of the illumination sources 22 may have a wide field of illumination, for example, a spatial cone having an angle of illumination of 90-180 degrees. Some illumination sources may have a narrow field of illumination, for example, a spatial cone having an angle of illumination of 1-90 degrees.

In accordance with one embodiment, the in-vivo imaging device 12 may include four illumination sources 22 at one of its ends, two of the illumination sources may be, for example, wide field illumination sources 22a and the other two illumination sources may be, for example, narrow field illumination sources 22b. In some embodiments, the two narrow field illumination sources 22b may have the same illumination fields, e.g., illumination field of 50° cone beam. In some embodiments, the two narrow field illumination sources 22b may have different narrow illumination fields, e.g. one illumination source may have, for example, illumination field of 70° while another narrow-field illumination source may have, for example, illumination field of 30°. In some embodiments, the two wide field illumination sources 22a may have the same illumination fields, e.g., illumination field of 120°. In some embodiments, the two wide field illumination sources 22a may have different illumination fields, e.g., one illumination source may have, for example, illumination filed of 100° while another illumination source may have, for example, illumination filed of 150°.

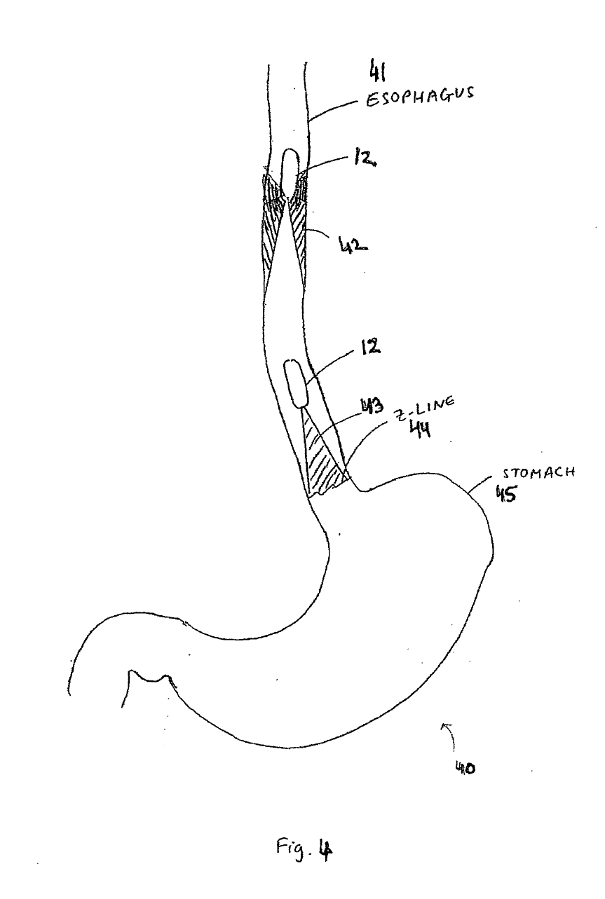

Reference is now made to FIG. 3, showing an illustrative schematic side view of an in-vivo imaging device 12 with illumination sources at both ends or proximal to both ends in accordance with embodiments of the present invention. In-vivo imaging device 12 may have illumination sources at both of its ends, allowing it to capture images in both a forward and rearward direction, relative to the direction of motion, as it traverses the gastrointestinal tract or other body lumens of a patient. The illumination sources proximal to one end of the in-vivo imaging device 12 may be wide field illumination sources 22a and the illumination sources proximal to the other end may be narrow field illumination sources 22b. In some embodiments, in-vivo imaging device 12 may have both narrow field illumination sources and wide field illumination sources proximal to both ends. In some embodiments, illumination sources 22 may include plurality of illumination sources each illumination source may have a different illumination range. The illumination sources may be controlled, for example, by control unit 23. For example, narrow field illumination sources 22b may have an illumination field with a range of 10°-70°, while wide field illumination sources 22a may have an illumination field with a range of 80°-150°. A control signal, for example, from control unit 23, may activate illumination sources 22a and 22b alternatively. Other illumination control schemes are also possible.

According to other embodiments imaging device 12 may have other shapes and the illumination sources need not be located at an end of the device. Rather they may illuminate through a side window or a window located at another location.

In one embodiment, each source may be selectively operable, and may illuminate a target area during different time periods. The target area may be an area of the gastrointestinal tract or other body lumens or cavities of the patient. The time periods of operating of each illumination source may be separate, or may be overlapping. In another embodiment, the sources 22a and 22b may illuminate simultaneously. The images obtained while different illumination fields are activated may depict different perspectives of a viewing site. The shadows caused by protrusions and irregularities in the surface of the target area, and the shading and coloring of the surface topography may differ under each of the illumination fields. For example, the shadows vary in size and direction depending on the field of illumination, e.g., the angle of the illumination from the illumination source.

In alternate embodiments, rather than selectively operating illumination sources to be completely on or completely off, certain sources may be dimmed or have their illumination fields varied at certain times, thereby producing effects enabling the capture and/or the analysis and understanding of surface orientation. Furthermore, in certain embodiment, the various illumination sources may provide different spectra of illumination (e.g., red, green or blue spectra, infra-red spectra or UV spectra).

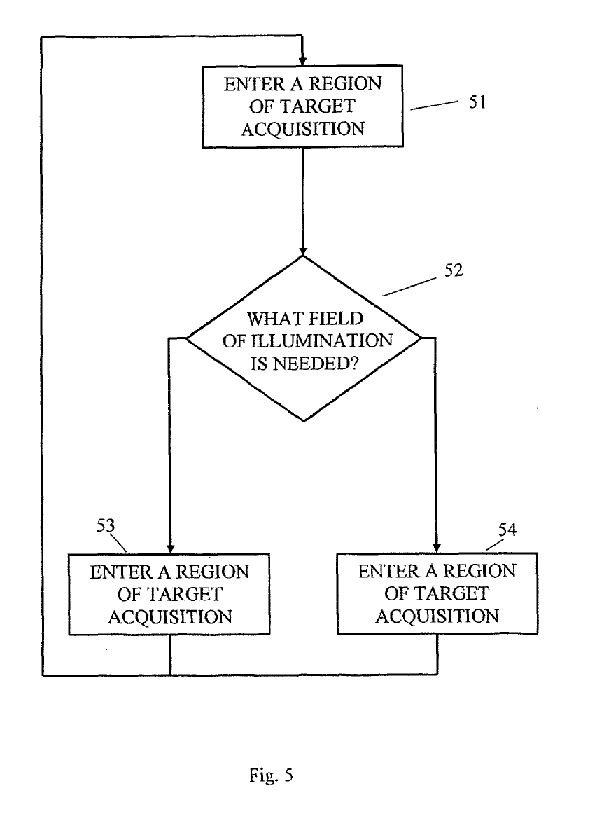

Reference is now made to FIG. 4, showing an illustration of an in-vivo imaging device in the GI tract in accordance with embodiments of the present invention. The in vivo device 12 may pass through the GI tract 40. In some embodiments under certain conditions, it may be advantageous to use illumination sources having a wide field of illumination 42, e.g., a field of illumination of about 120° which may be aimed asides of the longitudinal axis of on-vivo device 12. For example, when viewing the esophagus 41 it may be advantageous to illuminate relatively large target areas or sites, as shown by the in-vivo imaging device 12 at the upper location in FIG. 4, where two illumination sources having a wide field of illumination are seen. Under certain conditions, it may be advantageous for the illumination sources to have a narrow field of illumination 43, for example, a field of illumination of about 50°. For example, when viewing the Z-line 44, as depicted by the in-vivo imaging device 12 located at the lower location in FIG. 4. The Z-line marks the transition between the esophagus 41 and the stomach 45. In some embodiments only one illumination source having a narrow field of illumination 43 may be used. For example, In order to view the whole of Z-line 44 the imager 18 (shown in FIG. 1) has to be relatively distant from Z-line 44, and in order to have lighting of a sufficient intensity, the field of illumination of each illumination source should be relatively narrow so as to concentrate the illumination on Z-line 44 and not in addition on the surrounding inner wall of the esophagus 41, thus ensuring sufficient light-density at the area of interest.

In some embodiments of the present invention illumination sources 22a or 22b may be activated based on the illumination field required with relation to a specific target area, for example, wide field illumination sources 22b may be turned on when viewing the internal esophagus wall 41, and the narrow field illumination sources 22a may be turned on when, for example, viewing Z-line 44.

In some embodiments the wide field and narrow field illumination sources 22a, 22b respectively, may be operated and controlled by an external signal, for example, given by an operator, such as a health professional who may watch the a video movie of the data stream of images captured by the imager 18 of the in-vivo imaging device 12 as it traverses the gastrointestinal tract or other body lumens of a patient. In other embodiment's illumination sources 22a, 22b may be operated alternately at a periodically time intervals. For example, illumination sources 22a, 22b may be operated by a known time-dependent pattern which is based on the time intervals in which the in-vivo device 12 is located at different positions in the GI tract.

Reference is now made to FIG. 5 which is a flowchart depicting a method according to an embodiment of the invention. In vivo device 12 may traverse the gastrointestinal tract or other body lumens of a patient in order to capture in vivo images of target areas and may enter a region of target acquisition as indicated in block 51. The target area may have certain physical characteristics which may require a certain field of illumination. As is indicated in block 52, a decision may be made as to which field of illumination or angle of illumination is needed in order for imager 18 to capture images with certain characteristics such as high quality images, images with a certain angle of shading, images with a specific brightness or other characteristics.

If a wide field of illumination is needed a wide field of illumination sources may be used for lighting as indicated in block 53, and if, for example, the target area may be narrow and/or the in vivo device may be in proximity to the target area, for example, while the in vivo device 12 may be in the esophagus.

If a narrow field of illumination is needed a narrow field of illumination sources may be used for lighting as indicated in block 54. For example the target area may require a narrow field of illumination in order for imager 18 to capture images with certain characteristics, such as images with a high quality, images with a specific angle of shading, images with a specific brightness or other characteristics. For example, in order to view the Z-line 44 the imager 18 has to be relatively distant from Z-line 44, and in order to have lighting of sufficient intensity, the narrow field illumination source(s) may be used for lighting as indicated in block 54. The field of illumination of each illumination source should be relatively narrow so as to concentrate the illumination on Z-line 44 and not in addition on the surrounding inner wall of the esophagus.

While the present invention has been described with reference to one or more specific embodiments, the description is intended to be illustrative as a whole and is not to be construed as limiting the invention to the embodiments shown. It is appreciated that various modifications may occur to those skilled in the art that, while not specifically shown herein, are nevertheless within the scope of the invention.

What is claimed is:

1. An in vivo imaging device comprising:
an imager; and
a plurality of illumination sources wherein at least one illumination source has a first field of illumination and at least second illumination source has a second field of illumination.

2. The device according to claim 1, wherein said first field of illumination has a spatial illumination angle of substantially 1-90 degrees and wherein said second field of illumination has a spatial illumination angle of substantially 91-180 degrees.

3. The device according to claim 1 comprising a controller configured to control the illumination sources in a selective manner.

4. The device according to claim 3, wherein the selective manner comprises activating a first illumination source to illuminate a first target area and activating a second illumination source to illuminate a second target area.

5. The device of claim 4, wherein said first target area has different characteristics than said second target area.

6. The device according to claim 1, further comprising a transmitter for transmitting image data.

7. The device according to claim 1, wherein the illumination sources are located proximal to one end of the device.

8. The device according to claim 1, wherein the illumination sources are located proximal to both ends of the device.

9. A method providing an in vivo capturing of images, the method comprising:
illuminating an in vivo target area with at least one illumination source having a first field of illumination or with at least a second illumination source having a second field of illumination.

10. The method of claim 9, wherein said first field of illumination has a spatial illumination angle of 1-90 degrees and wherein said second field of illumination has a spatial illumination angle of 91-180 degrees.

11. The method of claim 9 further comprising activating a first illumination source to illuminate a first target area and activating a second illumination source to illuminate a second target area.

12. The method of claim 11, wherein said first target area has different characteristics than said second target area.

13. The method of claim 11 further comprising controlling activating said at least first illumination source or said at least second illumination source.
